(19) United States
(12) Patent Application Publication
(10) Pub. No.: US 2005/0282707 A1
(43) Pub. Date: Dec. 22, 2005

(54) Pyridinylisoxazoles and their use as herbicides

(75) Inventors: Andreas van Almsick, Karben (DE); Lothar Willms, Hofheim (DE); Thomas Auler, Bad Soden (DE); Martin Hills, Idstein (DE); Heinz Kehne, Hofheim (DE); Dieter Feucht, Eschborn (DE); Dorothee Hoischen, Dusseldorf (DE)

Correspondence Address: CONNOLLY BOVE LODGE & HUTZ, LLP, P O BOX 2207, WILMINGTON, DE, 19899, US

(73) Assignee: Bayer CropScience GmbH, Frankfurt am Main (DE)

(21) Appl. No.: 11/155,979

(22) Filed: Jun. 17, 2005

(30) Foreign Application Priority Data
Jun. 17, 2004 (DE) 10 2004 029 309.0

(51) Int. Cl. A01N 43/40; C07D 413/02

(52) U.S. Cl. 504/252; 546/272.1

(57) ABSTRACT

A description is given of pyridinylisoxazoles of the formula (I) or salts thereof. 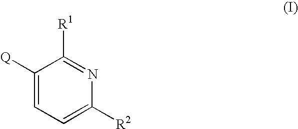
In this formula (I) Q is one of the radicals Q1, Q2 or Q3; 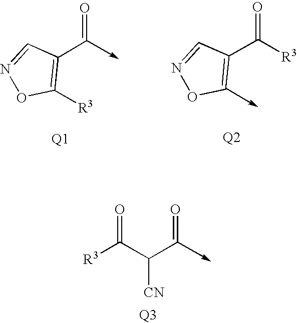
R¹, R² and R³ are various radicals, and n is 0 to 2.

The invention pertains to the technical field of herbicides, particularly that of herbicides for selectively controlling broadleaf and gramineous weeds in crops of useful plants.

From a variety of publications it is known that certain isoxazoles and diketonitriles which are substituted by a benzoyl or heteroaroyl radical possess herbicidal properties. For instance, EP 0 588 357 disclosed numerous 4-heteroaroyl-isoxazoles. Included in the description therein are some 4-pyridinyl-oyl-isoxazoles, in which the linkage is in position 3 of the pyridine ring, and the pyridine ring carries a further substituent in position 2. EP 0 524 018 describes 5-aryl-isoxazoles with a carbonyl group in position 4, where one of the possible meanings of aryl is pyridinyl. 5-(3-Pyridinyl)isoxazoles, in contrast, are not disclosed.

The known compounds, however, frequently exhibit an inadequate herbicidal activity or inadequate tolerance by crop plants. It is an object of the present invention, therefore, to provide herbicidally active compounds whose herbicidal properties are improved—improved, that is, over those of the prior art compounds.

It has now been found that 4-(3-pyridinyl-oyl)isoxazoles, 5-(3-pyridinyl)isoxazoles or (3-pyridinyl-oyl)-3-oxopropionitriles whose pyridine ring is substituted by a further radical in position 6 are especially suitable as herbicides. The present invention accordingly provides compounds of the formula (I) or salts thereof 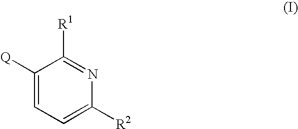
in which
Q is one of the radicals Q1, Q2 or Q3; 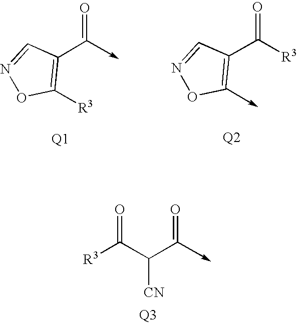
R1 is methyl;
R2 is Cl, Br, CF₃, S(O)ₙCH₃ or S(O)ₙC₂H₅;
R3 is methyl, ethyl, isopropyl, cyclopropyl or tertiary-butyl; and
n is 0, 1 or 2.

Where Q is Q3 the compounds of the formula (I) according to the invention, depending on external conditions, such as solvent and pH, may occur in different tautomeric structures:

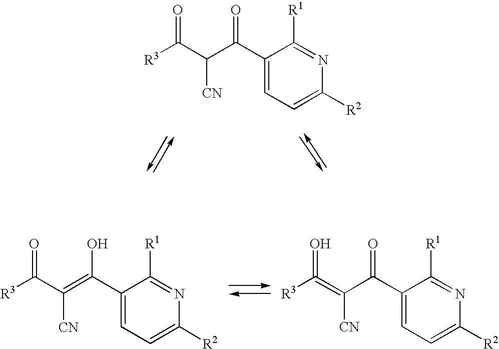

Depending on the nature and linkage of the substituents the compounds of the formula (I) may be present in the form of stereoisomers. Where, for example, there are one or more asymmetric carbon atoms, enantiomers and diastereomers may occur. Stereoisomers can be obtained from the as-prepared mixtures by standard separation methods, such as chromatographic separation methods, for example. Stereoisomers can also be prepared selectively by using stereoselective reactions and employing optically active starting materials and/or auxiliaries. The invention also provides all stereoisomers and mixtures thereof that, while embraced by the formula (I), are not defined specifically.

Preferred compounds of the formula (I) are those in which Q is Q1.

Particularly preferred compounds of the formula (I) are those in which R³ is cyclopropyl.

In all formulae below, the substituents and symbols, unless defined otherwise, have the same definition as described under formula (I).

From Pesticide Science 50, 83-84 (1997) it is know that certain isoxazoles—similar to the substructures Q1 and Q2—are able under certain conditions to undergo rearrangement to form an open-chain 3-oxopropionitrile—similar to substructure Q3.

The compounds of the formula (I) according to the invention in which Q is Q1 or Q2 can be prepared, for example, according to Scheme 1 by acylating the β-keto esters of the formula A1 which are known per se (Y. Oikawa et al., JOC 43, 2087, 1978) with a pyridine carboxylic acid derivative of the formula A2 in which T is chlorine to give an ester of the formula A3. Subsequent acid cleavage, by means for example of heating in the presence of trifluoroacetic acid or of heating in the presence of p-toluenesulfonic acid in toluene, gives a 1,3-diketone of the formula A4, which is reacted with an orthocarboxylic ester or with a carboxamide acetal to give a compound of the formula A5 in which L is a leaving group such as ethoxy or N,N-dimethylamino. Finally, base-catalyzed reaction with hydroxylamine and subsequent chromatographic separation give the compounds (I) of the invention in which Q is a radical of the formula Q1 or Q2.

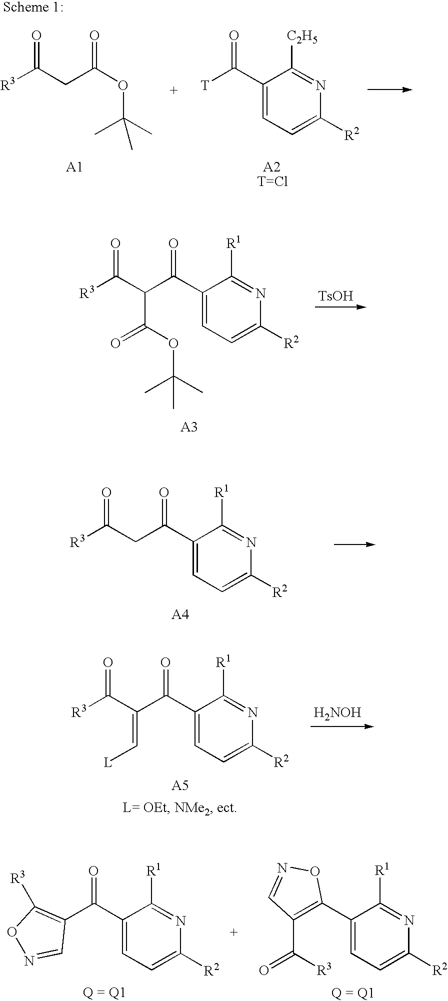

The compounds of the formula (I) according to the invention in which Q is Q3 can be obtained, for example, directly from the compounds of the formula (I) according to the invention where Q=Q1 or Q2 by reaction in the presence of a base such as NEt₃ (Scheme 2), or by reacting the magnesium enolate of a cyano ketone of the formula A6 with a pyridine-carboxylic acid derivative of the formula A2 (T=Cl) (Scheme 3).

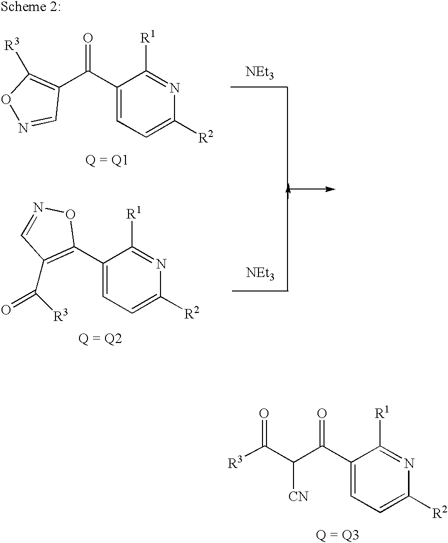

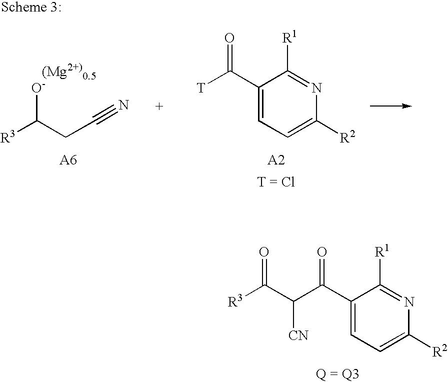

The pyridinecarboxylic acid derivatives of the formula A2 in which T is chlorine can be prepared in conventional manner by reacting the pyridine-carboxylic acids of the formula A2 (T=OH) with thionyl chloride or oxalyl chloride.

The pyridinecarboxylic acids of the formula A2 (T=OH) can be prepared in conventional manner by acidic or basic hydrolysis from the corresponding esters of the formula A2 (T=C₁-C₄ alkoxy).

The pyridinecarboxylic acids of the formula A2 are known or can be prepared in conventional manner.

The compounds of the formula (I) according to the invention have an excellent herbicidal activity against a broad spectrum of economically important monocotyledonous and dicotyledonous weed plants. The active substances provide effective control even of perennial weeds which produce shoots from rhizomes, root stocks or other perennial organs and which cannot be easily controlled. In this context, it generally does not matter whether the substrates are applied before sowing, pre-emergence or post-emergence. Some representative of the monocotyledonous and dicotyledonous weed flora, which can be controlled by the compounds according to the invention may be mentioned individually as examples, but this is not to be taken to mean a restriction to certain species. The monocotyledonous weed species which are controlled well are, for example, Avena, Lolium, Alopecurus, Phalaris, Echinochloa, Digitaria, Setaria and Cyperus species from the annual group, and Agropyron, Cynodon, Imperata and Sorghum or else perennial Cyperus species amongst the perennial species. In the case of dicotyledonous weed species, the spectrum of action extends to species such as, for example, Galium, Viola, Veronica, Lamium, Stellaria, Amaranthus, Sinapis, Ipomoea, Sida, Matricaria and Abutilon from the annual group, and Convolvulus, Cirsium, Rumex and Artemisia among the perennial weeds. Harmful plants which are found under the specific culture conditions of rice, such as, for example, Echinochloa, Sagittaria, Alisma, Eleocharis, Scirpus and Cyperus, are also controlled outstandingly well by the active substances according to the invention. If the compounds according to the invention are applied to the soil surface prior to germination, then either emergence of the weed seedlings is prevented completely, or the weeds grow until they have reached the cotyledon stage but growth then comes to a standstill and, after a period of three to four weeks, the plants eventually die completely. When the active substances are applied post-emergence to the green parts of the plants, growth also stops drastically very soon after the treatment, and the weeds remain at the growth stage of the time of application, or, after a certain period of time, they die completely so that in this way competition by the weeds, which is detrimental for the crop plants, is thus eliminated at a very early stage and in a sustained manner. In particular, the compounds according to the invention have an outstanding action against Apera spica venti, Chenopodium album, Lamium purpureum, Polygonum convolvulus, Stellaria media, Veronica hederifolia, Veronica persica and Viola tricolor.

The compounds according to the invention have an outstanding herbicidal activity against monocotyledonous and dicotyledonous weeds, and yet crop plants of economically important crops such as, for example, wheat, barley, rye, rice, maize, sugar beet, cotton and soya suffer only negligible damage, if any, in particular, they are outstandingly well tolerated in cereals, such as wheat, barley and maize, in particular wheat. This is why the present compounds are highly suitable for the selective control of unwanted vegetation in stands of agriculturally useful plants or of ornamentals.

Owing to their herbicidal properties, the active substances can also be employed for controlling weed plants in crops of genetically modified plants which are known or are yet to be developed. As a rule, the transgenic plants are distinguished by particularly advantageous properties, for example by resistances to certain pesticides, especially certain herbicides; by resistances to plant diseases or causative organisms of plant diseases, such as certain insects or microorganisms such as fungi, bacteria or viruses. Other particular properties concern for example the harvested material with regard to quantity, quality, shelf life, composition and specific constituents. Thus, transgenic plants are known which have an increased starch content or whose starch quality has been modified, or those whose fatty acid composition in the harvested material is different.

The compounds of the formula (I) according to the invention or their salts are preferably employed in economically important transgenic crops of useful plants and ornamentals, for example cereals such as wheat, barley, rye, oats, millet, rice, cassava and maize, or else crops of sugar beet, cotton, soya, oilseed rape, potato, tomato, pea and other vegetables. The compounds of the formula (I) can preferably be employed as herbicides in crops of useful plants which are resistant, or have been genetically modified to be resistant, to the phytotoxic effects of the herbicides.

Conventional routes for the generation of novel plants which have modified properties compared with existing plants are, for example, traditional breeding methods and the generation of mutants. Alternatively, novel plants with modified properties can be generated with the aid of recombinant methods (see, for example, EP-A-0221044, EP-A-0131624). For example, several cases of the following have been described:
recombinant modifications of crop plants for the purposes of modifying the starch synthesized in the plants (e.g. WO 92/11376, WO 92/14827 WO 91/19806),
transgenic crop plants which exhibit resistances to certain herbicides of the glufosinate type (cf. eg. EP-A-0242236, EP-A-242246), glyphosate type (WO 92/00377) or of the sulfonylurea type (EP-A-0257993, U.S. Pat. No. 5,013,659)
transgenic crop plants, for example cotton, with the ability to produce Bacillus thuringienis toxins (Bt toxins), which make the plants resistant to certain pests (EP-A-0142924, EP-A-0193259),
transgenic crop plants with a modified fatty acid composition (WO 91/13972),

A large number of techniques in molecular biology, with the aid of which novel transgenic plants with modified properties can be generated, are known in principle; see, for example, Sambrook et al., 1989, Molecular Cloning, A Laboratory Manual, 2nd Ed., Cold Spring Harbor Laboratory Press, Cold Spring Harbor, N.Y.; or Winnacker “Gene und Klone” [Genes and Clones], VCH Weinheim 2nd Edition 1996 or Christou, “Trends in Plant Science” 1 (1996) 423-431.

To carry out such recombinant manipulations, nucleic acid molecules can be introduced into plasmids which permit a mutagenesis or a sequence alteration by recombination of DNA sequences. With the aid of the abovementioned standard processes, it is possible, for example, to carry out base substitutions, to remove part sequences or to add natural or synthetic sequences. The fragments can be provided with adapters or linkers to link the DNA fragments to each other.

Plant cells with a reduced activity of a gene product can be obtained, for example, by expressing at least one corresponding antisense RNA, a sense RNA for achieving a cosuppression effect, or the expression of at least one suitably constructed ribozyme which specifically cleaves transcripts of the abovementioned gene product.

To this end, it is possible, on the one hand, to use DNA molecules which encompass all of the coding sequence of a gene product including any flanking sequences which may be present, but also DNA molecules which only encompass portions of the coding sequence, it being necessary for these portions to be so long as to cause an antisense effect in the cells. Another possibility is the use of DNA sequences which have a high degree of homology with the coding sequences of a gene product, but are not completely identical.

When expressing nucleic acid molecules in plants, the protein synthesized may be localized in any desired compartment of the plant cell. However, to achieve localization in a particular compartment, the coding region can, for example, be linked to DNA sequences which ensure localization in a particular compartment. Such sequences are known to the skilled worker (see, for example, Braun et al., EMBO J. 11(1992), 3219-3227, Wolter et al., Proc. Natl. Acad. Sci. USA 85 (1988), 846-850; Sonnewald et al., Plant J. 1 (1991), 95-106).

The transgenic plant cells can be regenerated by known techniques to give intact plants. In principle, the transgenic plants can be plants of any desired plant species, i.e., both monocotyledonous and dicotyledonous plants.

Thus, transgenic plants can be obtained which exhibit modified properties owing to the overexpression, suppression or inhibition of homologous (i.e., natural) genes or gene sequences or expression of heterologous (i.e., foreign) genes or gene sequences.

When using the active substances according to the invention in transgenic crops, effects are frequently observed—in addition to the effects against weed plants to be observed in other crops—which are specific for the application in the transgenic crop in question, for example a modified or specifically widened controllable weed spectrum, modified application rates which may be employed for the application, preferably good combining ability with the herbicides to which the transgenic crop is resistant, and an effect on the growth and yield of the transgenic crop plants. The invention therefore also relates to the use of the compounds according to the invention as herbicides for controlling harmful plants in transgenic crop plants.

The substances according to the invention additionally have outstanding growth-regulatory properties in crop plants. They engage in the plants' metabolism in a regulatory fashion and can thus be employed for the targeted influencing of plant constituents and for facilitating harvesting, such as, for example, by triggering desiccation and stunted growth. Moreover, they are also suitable for generally controlling and inhibiting unwanted vegetative growth without destroying the plants in the process. Inhibiting the vegetative growth plays an important role in many monocotyledonous and dicotyledonous crops, allowing lodging to be reduced or prevented completely.

The compounds according to the invention can be employed in the form of wettable powders, emulsifiable concentrates, sprayable solutions, dusts or granules in the customary preparations. The invention therefore further relates to herbicidal compositions comprising compounds of the formula (I). The compounds of the formula (I) can be formulated in various ways, depending on the prevailing biological and/or chemico-physical parameters. Examples of suitable formulations which are possible are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for spreading and soil application, granules (GR) in the form of microgranules, spray granules, coated granules and adsorption granules, water-dispersible granules (WG), water-soluble granules (SG), ULV formulations, microcapsules and waxes. These individual formulation types are known in principle and are described, for example, in Winnacker-Küchler, “Chemische Technologie” [Chemical Technology], Volume 7, C. Hauser Verlag Munich, 4th Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials; surfactants, solvents and further additives, are likewise known and are described, for example, in: Watkins, “Handbook of Insecticide Dust Diluents and Carriers”, 2nd Ed., Darland Books, Caldwell N.J., H. v. Olphent “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964, Schönfeldt, “Grenztlächenaktive Äthylenoxidaddukte” [Surface-active ethylene oxide adducts], Wiss Verlagsgesell., Stuttgart 1976; Winnacker-Küchler, “Chemische Technologie”, Volume 7, C. Hauser Verlag Munich, 4th Ed. 1986.

Wettable powders are preparations which are uniformly dispersible in water and which, in addition to the active substance, also contain ionic and/or nonionic surfactants (wetters, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium 2,2′-dinaphthylmethane-6,6′-disulfonate, sodium lignosulfonate, sodium dibutylnaphthalenesulfonate or else sodium oleoylmethyltauride, in addition to a diluent or inert substance. To prepare the wettable powders, the herbicidal active substances are ground finely, for example in customary equipment such as hammer mills, blowing mills and air-jet mills, and simultaneously or subsequently mixed with the formulation auxiliaries.

Emulsifiable concentrates are prepared by dissolving the active substance in an organic solvent, such as butanol, cyclohexanone, dimethylformamide, xylene or else higher-boiling aromatics or hydrocarbons or mixtures of the organic solvents with addition of one or more ionic and/or nonionic surfactants (emulsifiers). Examples of emulsifiers which can be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active substance with finely divided solid materials, for example talc, natural clays such as kaolin, bentonite and pyrophyllite, or diatomaceous earth.

Suspension concentrates can be water based or oil based. They can be prepared for example by wet-grinding by means of customary bead mills, if appropriate with addition of surfactants, as have already been mentioned for example above in the case of the other formulation types.

Emulsions, for example oil-in-water emulsions (EW), can be prepared for example by means of stirrers, colloid mills and/or static mixers using aqueous organic solvents and, if appropriate, surfactants as have already been mentioned for example above in the case of the other formulation types.

Granules can be prepared either by spraying the active substance onto adsorptive, granulated inert material or by applying active substance concentrates to the surface of carriers such as sand, kaolinites or granulated inert material with the aid of tackifiers, for example polyvinyl alcohol, sodium polyacrylate or else mineral oils. Suitable active substances can also be granulated in the fashion which is conventional for the production of fertilizer granules, if desired as a mixture with fertilizers.

Water-dispersible granules are generally prepared by customary methods such as spray drying, fluidized-bed granulation, disk granulation, mixing with high-speed stirrers and extrusion without solid inert material.

To prepare disk granules, fluidized-bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 et seq.; “Perry's Chemical Engineers Handbook”, 5th Ed., McGraw-Hill, New York 1973, pp. 8-57.

For further details on the information of crop protection products see for example G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical preparations comprise 0.1 to 99% by weight, in particular 0.1 to 95% by weight, of active of the formula (I). In wettable powders, the active substance concentration is, for example, approximately 10 to 90% by weight, the remainder to 100% by weight being composed of customary formulation constituents. In the case of emulsifiable concentrates, the active substance concentration can amount to approximately 1 to 90, preferably 5 to 80% by weight. Formulations in the form of dusts comprise 1 to 30% by weight of active substance, preferably in most cases 5 to 20% by weight of active substance, and sprayable solutions comprise approximately 0.05 to 80, preferably 2 to 50% by weight of active substance. In the case of water-dispersible granules, the active substance content depends partly on whether the active compound is in liquid or solid form and on the granulation auxiliaries, fillers and the like which are being used. In the case of the water-dispersible granules, for example, the active substance content is between 1 and 95% by weight, preferably between 10 and 80% by weight.

In addition, the active substance formulations mentioned comprise, if appropriate, the stickers, wetters, dispersants, emulsifiers, penetrants, preservatives, antifreeze agents, solvents, fillers, carriers, colorants, antifoams, evaporation inhibitors, and pH and viscosity regulators which are conventional in each case.

Based on these formulations, it is also possible to prepare combinations with other pesticidally active substances such as, for example, insecticides, acaricides, herbicides, fungicides, and with safeners, fertilizers and/or growth regulators, for example in the form of a readymix or a tank mix.

Active substances which can be employed in combination with the active substances according to the invention in mixed formulations or in a tank mix are, for example, known active substances as are described, for example, in Weed Research 26, 441-445 (1986) or “The Pesticide Manual”, 11th edition, The British Crop Protection Council and the Royal Soc. of Chemistry, 1997 and literature cited therein. Known herbicides which are to be mentioned, and can be combined with the compounds of the formula (I), are, for example, the following active substances (note: the compounds are either designated by the common name according to the International Organization for Standardization (ISO) or using the chemical name, if appropriate together with a customary code number):
acetochlor; acifluorfen; aclonifen; AKH 7088, i.e. [[[1-[5-[2-chloro-4-(trifluoromethyl)-phenoxy]-2-nitrophenyl]-2-methoxyethylidene]amino]oxy] acetic acid and its methyl ester; alachlor; alloxydim; ametryn; amidosulfuron; amitrol; AMS, i.e., ammonium sulfamate; anilofos; asulam; atrazine; azimsulfurone (DPX-A8947); aziprotryn; barban; BAS 516 H, i.e., 5-fluoro-2-phenyl-4H-3,1-benzoxazin-4-one; benazolin; benfluralin; benfuresate; bensulfuron-methyl; bensulide; bentazone; benzofenap; benzofluor; benzoylprop-ethyl; benzthiazuron; bialaphos; bifenox; bromacil; bromobutide; bromofenoxim; bromoxynil; bromuron; buminafos; busoxinone; butachlor; butamifos; butenachlor; buthidazole; butralin; butylate; cafenstrole (CH-900); carbetamide; cafentrazone (ICI-A0051); CDAA, i.e. 2-chloro-N,N-di-2-propenylacetamide; CDEC, i.e. 2-chloroallyl diethyldithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron ethyl; chlornitrofen; chlorotoluron; chloroxuron; chlorpropham; chlorsulfuron; chlorthal-dimethyl; chlorthiamid; cinmethylin; cinosulfuron; clethodim; clodinafop and its ester derivatives (for example clodinafop-propargyl); clomazone; clomeprop; cloproxydim; clopyralid; cumyluron (JC 940); cynazine; cycloate; cyclosulfamuron (AC 104); cycloxydim; cycluron; cyhalofop and its ester derivatives (for example butyl ester, DEH-112); cyperquat; cyprazine; cyprazole; daimuron; 2,4-DB; dalapon; desmedipham; desmetryn; di-allate; dicamba; dichlobenil; dichlorprop; diclofop and its esters such as diclofop-methyl, diethatyl; difenoxuron; difenzoquat; diflufenican; dimefuron dimethachlor; demethametryn; dimethenamid (SAN-582H); dimethazone, clomazon; dimethipin dimetrasulfuron, dinitramine; dinoseb; dinoterb; diphenamid; dipropetryn; diquat; dithiopyr; diuron; DNOC; eglinazine-ethyl; EL 77, i.e., 5-cyano-1-(1,1-dimethyl)-N-methyl-1H-pyrazole-4-carboxamide; endothal; EPTC; esprocarb; ethalfluralin; ethametsulfuron-methyl; ethidimuron; ethiozin; ethofurnesate; F5231 i.e. N-[2-chloro-4-fluoro-5-[4-(3-fluoropropyl)-4,5-dihydro-5-oxo-1H-tetrazol-1-yl]phenyl]ethanesulfonamide; ethoxyfen and its esters (for example ethyl ester, HN-252); etobenzanid (HW 52); fenoprop; fenoxan, fenoxaprop and fenoxaprop-P and their esters, for example fenoxaprop-P-ethyl and fenoxaprop-ethyl; fenoxydim; fenuron; flamprop-methyl; flazasulfuron; fluazifop and fluazifop-P and their esters, for example fluazifop-butyl and fluazifop-P-butyl; fluchloralin; flumetsulam; flumeturon; flumiclorac and its esters (for example pentylester, S-23031); flumioxazin (S-482); flumipropyn; flupoxam (KNW-739); fluorodifen; fluoroglycofen-ethyl; flupropacil (UBIC-4243); fluridone; flurochloridone; fluroxypyr; flurtamone; fomesafen; fosamine; furyloxyfen; glufosinate; glyphosate; halosafen; halosulfuron and its esters (for example methyl ester, NC-319); haloxyfop and its esters; haloxyfop-P (=R-haloxyfop) and its esters; hexazinone; imazapyr; imazamethabenz-methyl; imazaquin and salts such as the ammonium salt; ioxynil; imazethamethapyr; imazethapyr; imazosulfuron; isocarbamid; isopropalin; isoproturon; isouron; isoxaben; isoxapyrifop; karbutilate; lactofen; lenacil; linuron; MCPA; MCPB; mecoprop; mefenacet; mefluidid; metamitron; metazachlor; metham; methabenzthiazuron; methazole; methoxyphenone; methyldymron; metabenzuron, methobenzuron; metobromuron; metolachlor; metosulam (XRD 511); metoxuron; metribuzin; metsulfuron-methyl; MH; molinate; monalide; monolinuron; monuron; monocarbamide dihydrogensulfate; MT 128, i.e., 6-chloro-N-(3-chloro-2-propenyl)-5-methyl-N-phenyl-3-pyridazinamine; MT 5950, i.e., N-[3-chloro-4-(1-methylethyl)phenyl]-2-methylpentanamide; naproanilide; napropamide; naptalam; NC 310, i.e., 4-(2,4-dichlorobenzoyl)-1-methyl-5-benzyloxypyrazole; neburon; nicosulfuron, nipyraclophen; nitralin; nitrofen; nitrofluorfen; norflurazon; orbencarb; oryzalin; oxadiargyl (RP-020630); oxadiazon; oxyfluorfen; paraquat; pebulate; pendimethalin; perfluidone; phenisopham; phenmedipham; picloram; piperophos; piributicarb; pirifenop-butyl; pretilachlor; primisulfuron-methyl; procyazine; prodiamine; profluralin; proglinazine-ethyl; prometon; prometryn; propachlor; propanil; propaquizafop and its esters; propazine; propham; propisochlor; propyzamide; prosulfalin; prosulfocarb; prosulfuron (CGA-152005); prynachlor; pyrazolinate; pyrazon; pyrazolsulfuron-ethyl; pyrazoxyfen; pyridate; pyrithiobac (KIH-2031); pyrozofop and its esters (for example propargyl ester); quinclorac; quinmerac; quinofop and its ester derivatives, quizalofop and quizalofop-P and their ester derivatives for example quizalofop-ethyl; quizalofop-P-tefuryl and their -ethyl; renriduron; rimsulfuron (DPX-E 9636); S 275, i.e., 2-[4-chloro-2-fluoro-5-(2-propynyloxy)phenyl]-4,5,6,7-tetrahydro-2H-indazole; secbumeton; sethoxydim; siduron; simazine; simetryn; SN 106279, i.e., 2-[[7-[2-chloro-4-(trifluoromethyl)phenoxy]-2-naphthalenyl]oxy]propanoic acid and its methyl ester; sulfentrazon (FMC-97285, F-6285); sulfazuron; sulfometuron-methyl; sulfosate (ICI-A0224); TCA; tebutam (GCP-5544); tebuthiuron; terbacil; terbucarb; terbuchlor; terbumeton; terbuthylazine; terbutryn; TFH 450, i.e., N,N-diethyl-3-[(2-ethyl-6-methylphenyl)sulfonyl]-1H-1,2,4-triazole-1-carboxamide; thenylchlor (NSK-850); thiazafluron; thiazopyr (Mon-13200); thidiazimin (SN-24085); thiobencarb; thifensulfuron-methyl; tiocarbazil; tralkoxydim; tri-allate; triasulfuron; triazofenamide; tribenuron-methyl; triclopyr; tridiphane; trietazine; trifluralin; triflusulfuron and esters (for example methyl ester, DPX-66037); trimeturon; tsitodef; vernolate; WL 110547, i.e., 5-phenoxy-1-[3-(trifluoromethyl)phenyl]-1H-tetrazole; UBH-509; D489; LS 82-556; KPP-300; NC-324; NC-330; KH-218; DPX-N8189; SC-0774; DOWCO-535; DK-8910; V-53482; PP-600; MBH-001; KIH-9201; ET-751; KIH-6127 and KIH-2023.

For use, the formulations, which are present in commercially available form, are diluted in the customary manner, for example using water in the case of wettable powders, emulsifiable concentrates, dispersions and water-dispersible granules. Preparations in the form of dusts, soil granules, granules for spreading and sprayable solutions are usually not diluted any further with other inert substances prior to use.

The required application rate of the compounds of the formula (I) varies with the external conditions such as, inter alia, temperature, humidity and the nature of the herbicide used. It can vary within wide limits, for example between 0.001 and 1.0 kg/ha or more of active substance, but it is preferably between 0.005 and 750 g/ha.

The examples which follow illustrate the invention.

### CHEMICAL EXAMPLES

### 1. (5-Cyclopropylisoxazol-4-yl)-(2-methyl-6-(trifluoromethyl)pyridine-3-yl)methanone (tabular example 1.44 and cyclopropyl{5-[2-methyl-6-(trifluoromethyl)pyridine-3-yl]isoxazol-4-yl}methanone (tabular example 2.44)

### a) 1-Cyclopropyl-3-[2-methyl-6-(trifluoromethyl)pyridine-3-yl]propane-1,3-dione 4.83 g (24 mmol) of 2-methyl-6-(trifluoromethyl)nicotinic acid were introduced in 150 ml of CH₂Cl₂, and one drop of DMF and 5.98 g (47 mmol) of oxalyl chloride were added. When the evolution of gas was at an end the mixture was heated under reflux for 3 h more and then concentrated. The residue was suspended in 100 ml of toluene. In a second batch, 4.34 g (24 mmol) of tert-butyl 3-cyclopropyl-3-oxopropanoate were introduced in 150 ml of methanol, and 0.57 g (24 mmol) of magnesium turnings and one drop of CCl₄ were added. The mixture was stirred at RT until all of the magnesium had reacted. Thereafter it was concentrated completely and the residue was dissolved in 150 ml of toluene. This solution was admixed dropwise with the above acid chloride solution and the combined system was then stirred at RT for 3 h. It was concentrated and the residue was taken up in 200 ml of EE, washed with water and dried over MgSO₄. The system was then concentrated again. The residue was dissolved in 100 ml of toluene, 0.1 g of p-toluenesulfonic acid was added, and the solution was heated under reflux for 2 h. Subsequently it was concentrated and the residue was taken up in 200 ml of EE, washed with water, dried over MgSO₄ and concentrated again.

Yield: 5.07 g (18.7 mmol) 78%, brown oil, 95% purity by HPLC ¹H NMR: δ[CDCl₃] 1.05 (m,2H), 1.25 (m,2H), 1.78 (m,1H), 2.78 (s,3H), 5.95 (s,1H), 7.58 (d,1H), 7.92 (d,1H)

### b) 1-Cyclopropyl-2[(dimethylamino)methylene]-3-[2-methyl-6-(trifluoromethyl)pyridin-3-yl]propane-1,3-dione

5.07 g (19 mmol) of 1-cyclopropyl-3-[2-methyl-6-(trifluoromethyl)pyridin-3-yl]propane-1,3-dione were, stirred together with 8.9 g (75 mmol) of N,N-dimethylformamide dimethyl acetal at RT for 3 h The mixture was subsequently concentrated and purified by chromatography.

Yield: 5.7 (17.5 mmol) 92%, brown oil, 95% purity by HPLC ¹H NMR: δ[CDCl₃] 0.65 (m,2H), 0.95 (m,2H), 1.82 (m,1H), 2.7 (s,3H), 2.82 (s,br,3H), 3.25 (s,br,3H), 7.45 (s,1H), 7.52 (d,1H), 7.75 (d,1H)

### c) (5-Cyclopropylisoxazol-4-yl)(2-methyl-6-(trifluoromethyl)pyridin-3-yl)methanone and cyclopropyl{5-[2-methyl-6-(trifluoromethyl)pyridin-3-yl]isoxazol-4-yl}methanone

1 g (2 mmol) of 1-cyclopropyl-2-[(dimethylamino)methylene]-3-[2-methyl-6-trifluoromethyl)pyridin-3-yl]propane-1,3,-dione was dissolved in 50 ml of ethanol and then 1.15 g (2 mmol) of hydroxylamine hydrochloride were added. The mixture was stirred at RT for 4 h. Thereafter it was concentrated and the residue was taken up in 100 ml of EE, washed with water, dried over MgSO₄ and concentrated again. The two products were separated by chromatography.

Yield: 235 mg (0.79 mmol) 40% (5-cyclopropylisoxazol-4-yl)[2-methyl-6-(trifluoromethyl)pyridin-3-yl]methanone as a yellowish resin ¹H NMR: δ[CDCl₃] 1.3 (m,2H), 1.4 (m,2H), 2.7 (m,1H), 2.7 (s,3H), 7.65 (d,1H), 7.85 (d,1H), 8.15 (s,1H) and 120 mg (0.41 mmol) 20% cyclopropyl{5-[2-methyl-6-trifluoromethyl)pyridine-3-yl]isoxazol-4-yl}methanone as a yellowish solid ¹H NMR: δ[CDCl₃] 1.0 (m,2H), 1.2 (m,2H), 2.05 (m,1H, 2.6 (s,3), 7.65 (s,1H) 7.98 (d,1H), 8.8 (s,1H)

### 2. 3-Cyclopropyl-2-{[2-methyl-6-methylsulfonyl)pyridin-3-yl]carbonyl}-3-oxopropanenitrile (tabular example 3.4)

1.48 g (5 mmol) of (5-cyclopropylisoxazol-4-yl)[2-methyl-6-(methylsulfonyl)pyridin-3-yl]methanone were dissolved in 100 ml of CH₂Cl₂, and 0.58 g (6 mmol) of NEt₃ was added. The mixture was stirred at RT for 2 h then washed with 10% strength sulfuric acid and saturated NaCl solution, dried over MgSO₄, and then concentrated.

Yield: 1.18 g (3.9 mmol) 78% as a yellowish oil ¹H NMR: δ[CDCl₃] 1.35 (m,2H) 1.5 (m,2H), 2.4 (m,1H), 2.75 (s,3H), 3.25 (s,3H), 8.05 (m,2H)

The examples listed in the tables below were prepared in analogy to methods specified above or are obtainable in analogy to methods specified above.

The abbreviations used have the following definitions:

| | | |
|---|---|---|
| Et = ethyl | Me = methyl | i-Pr = isopropyl |
| C-Pr = cyclopropyl | t-Bu = tertiary-butyl | m.p. = melting point |
| RT = room temperature | EE = ethyl ethanoate | R^{f} = retention value |
| | [ethyl ester of acetic | |
| | acid] | |

TABLE 1
Compounds of the formula (I) according to the invention in which the
substituents and symbols have the following definitions:

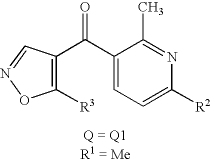

| No. | R² | R³ | Physical Data |
|---|---|---|---|
| 1.1 | SO₂Me | Me | |
| 1.2 | SO₂Me | Et | |
| 1.3 | SO₂Me | i-Pr | |
| 1.4 | SO₂Me | c-Pr | ¹H NMR: δ [CDCl₃] 1.3 (m, 2H), 1.4 (m, 2H), 2.68 (m, 1H), |
| | | | 2.7 (s, 3H), 3.28 (s, 3H), 7.92 (d, 1H), 8.05 (d, 1H), 8.17 |
| | | | (s, 1H) |
| 1.5 | SO₂Me | t-Bu | |
| 1.6 | SOMe | Me | |
| 1.7 | SOMe | Et | |
| 1.8 | SOMe | i-Pr | |
| 1.9 | SOMe | c-Pr | |
| 1.10 | SOMe | t-Bu | |
| 1.11 | SMe | Me | |
| 1.12 | SMe | Et | |
| 1.13 | SMe | i-Pr | |
| 1.14 | SMe | c-Pr | ¹H NMR: δ [CDCl₃] 1.15 (m, 2H), 1.28 (m, 2H), 2.55 |
| | | | (s, 3H), 2.57 (s, 3H), 2.57 (m, 1H), 7.01 (d, 1H), 7.45 |
| | | | (d, 1H), 8.15 (s, 1H) |
| 1.15 | SMe | t-Bu | |
| 1.16 | SO₂Et | Me | |
| 1.17 | SO₂Et | Et | |
| 1.18 | SO₂Et | i-Pr | |
| 1.19 | SO₂Et | c-Pr | R^{f}: 0.75 (EE) |
| 1.20 | SO₂Et | t-Bu | |
| 1.21 | SOEt | Me | |
| 1.22 | SOEt | Et | |
| 1.23 | SOEt | i-Pr | |
| 1.24 | SOEt | c-Pr | |
| 1.25 | SOEt | t-Bu | |
| 1.26 | SEt | Me | |
| 1.27 | SEt | Et | |
| 1.28 | SEt | i-Pr | |
| 1.29 | SEt | c-Pr | R^{f}: 0.8 (EE) |
| 1.30 | SEt | t-Bu | |
| 1.31 | Br | Me | |
| 1.32 | Br | Et | |
| 1.33 | Br | i-Pr | |
| 1.34 | Br | c-Pr | m.p.: 119-120° C. |
| 1.35 | Br | t-Bu | |
| 1.36 | Cl | Me | |
| 1.37 | Cl | Et | |
| 1.38 | Cl | i-Pr | |
| 1.39 | Cl | c-Pr | ¹H NMR: δ[CDCl₃] 1.25 (m, 2H), 1.38 (m, 2H), 2.62 (s, 3H), |
| | | | 2.65 (m, 1H), 7.3 (d, 1H), 7.65 (d, 1H), 8.18 (s, 1H) |
| 1.40 | Cl | t-Bu | |
| 1.41 | CF₃ | Me | |
| 1.42 | CF₃ | Et | |
| 1.43 | CF₃ | i-Pr | |
| 1.44 | CF₃ | c-Pr | |
| 1.45 | CF₃ | t-Bu | |

TABLE 2
Compounds of the formula (I) according to the invention in which the
substituents and symbols have the following definitions:

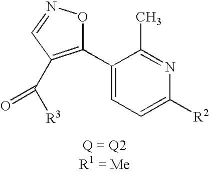

| No. | R² | R³ | Physical Data |
|---|---|---|---|
| 2.1 | SO₂Me | Me | |
| 2.2 | SO₂Me | Et | |
| 2.3 | SO₂Me | i-Pr | |
| 2.4 | SO₂Me | c-Pr | ¹H NMR: δ[CDl₃] 1.03 (m, 2H), 1.22 (m, 2H), 2.12 |
| | | | (m, 1H), 2.6 (s, 3H), 3.3 (s, 3H), 8.03 (d, 1H), 8.08 (d, 1H), |
| | | | 8.82 (s, 1H) |
| 2.5 | SO₂Me | t-Bu | |
| 2.6 | SOMe | Me | |
| 2.7 | SOMe | Et | |
| 2.8 | SOMe | i-Pr | |
| 2.9 | SOMe | c-Pr | |
| 2.10 | SOMe | t-Bu | |
| 2.11 | SMe | Me | |
| 2.12 | SMe | Et | |
| 2.13 | SMe | i-Pr | |
| 2.14 | SMe | c-Pr | ¹H NMR: δ[CDCl₃] 0.85 (m, 2H), 1.15 (m, 2H), 1.95 |
| | | | (m, 1H), 2.43 (s, 3H), 2.55 (s, 3H), 7.05 (d, 1H), 7.5 (d, 1H), |
| | | | 8.65 (s, 1H) |
| 2.15 | SMe | t-Bu | |
| 2.16 | SO₂Et | Me | |
| 2.17 | SO₂Et | Et | |
| 2.18 | SO₂Et | i-Pr | |
| 2.19 | SO₂Et | c-Pr | R^{f}: 0.75 (EE) |
| 2.20 | SO₂Et | t-Bu | |
| 2.21 | SOEt | Me | |
| 2.22 | SOEt | Et | |
| 2.23 | SOEt | i-Pr | |
| 2.24 | SOEt | c-Pr | |
| 2.25 | SOEt | t-Bu | |
| 2.26 | SEt | Me | |
| 2.27 | SEt | Et | |
| 2.28 | SEt | i-Pr | |
| 2.29 | SEt | c-Pr | R^{f}: 0.8 (EE) |
| 2.30 | SEt | t-Bu | |
| 2.31 | Br | Me | |
| 2.32 | Br | Et | |
| 2.33 | Br | i-Pr | |
| 2.34 | Br | c-Pr | |
| 2.35 | Br | t-Bu | |
| 2.36 | Cl | Me | |
| 2.37 | Cl | Et | |
| 2.38 | Cl | i-Pr | |
| 2.39 | Cl | c-Pr | R^{f}: 0.85 (EE) |
| 2.40 | Cl | t-Bu | |
| 2.41 | CF₃ | Me | |
| 2.42 | CF₃ | Et | |
| 2.43 | CF₃ | i-Pr | |
| 2.44 | CF₃ | c-Pr | |
| 2.45 | CF₃ | t-Bu | |

TABLE 3
Compounds of the formula (I) according to the invention in which the
substituents and symbols have the following definitions:

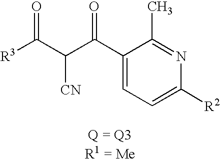

| No. | R² | R³ | Physical Data |
|---|---|---|---|
| 3.1 | SO₂Me | Me | |
| 3.2 | SO₂Me | Et | |
| 3.3 | SO₂Me | i-Pr | |
| 3.4 | SO₂Me | c-Pr | |
| 3.5 | SO₂Me | t-Bu | |
| 3.6 | SOMe | Me | |
| 3.7 | SOMe | Et | |
| 3.8 | SOMe | i-Pr | |
| 3.9 | SOMe | c-Pr | |
| 3.10 | SOMe | t-Bu | |
| 3.11 | SMe | Me | |
| 3.12 | SMe | Et | |
| 3.13 | SMe | i-Pr | |
| 3.14 | SMe | c-Pr | |
| 3.15 | SMe | t-Bu | |
| 3.16 | SO₂Et | Me | |
| 3.17 | SO₂Et | Et | |
| 3.18 | SO₂Et | i-Pr | |
| 3.19 | SO₂Et | c-Pr | |
| 3.20 | SO₂Et | t-Bu | |
| 3.21 | SOEt | Me | |
| 3.22 | SOEt | Et | |
| 3.23 | SOEt | i-Pr | |
| 3.24 | SOEt | c-Pr | |
| 3.25 | SOEt | t-Bu | |
| 3.26 | SEt | Me | |
| 3.27 | SEt | Et | |
| 3.28 | SEt | i-Pr | |
| 3.29 | SEt | c-Pr | |
| 3.30 | SEt | t-Bu | |
| 3.31 | Br | Me | |
| 3.32 | Br | Et | |
| 3.33 | Br | i-Pr | |
| 3.34 | Br | c-Pr | |
| 3.35 | Br | t-Bu | |
| 3.36 | Cl | Me | |
| 3.37 | Cl | Et | |
| 3.38 | Cl | i-Pr | |
| 3.39 | Cl | c-Pr | ¹H NMR: δ[CDCl₃] 1.3 (m, 2H), 1.45 (m, 2H), 2.4 (m, 1H), |
| | | | 2.65 (s, 3H), 7.3 (d, 1H), 7.8 (d, 1H), 17.5 (s, br, 1H) |
| 3.40 | Cl | t-Bu | |
| 3.41 | CF₃ | Me | |
| 3.42 | CF₃ | Et | |
| 3.43 | CF₃ | i-Pr | |
| 3.44 | CF₃ | c-Pr | ¹H NMR: δ[CDCl₃] 1.35 (m, 2H), 1.45 (m, 2H), 2.4 (m, 1H), |
| | | | 2.7 (s, 3H), 7.65 (d, 1H), 7.97 (d, 1H) |
| 3.45 | CF₃ | t-Bu | |

### B. FORMULATION EXAMPLES

### 1. Dust

A dust is obtained by mixing 10 parts by weight of a compound of the formula (I) and 90 parts by weight of talc as inert substance and comminuting the mixture in a hammer mill.

### 2. Dispersible Powder

A wettable powder which is readily dispersible in water is obtained by mixing 25 parts by weight of a compound of the formula (I), 64 parts by weight of kaolin-containing quartz as inert substance, 10 parts by weight of potassium ligninsulfonate and 1 part by weight of sodium oleoylmethyltauride as wetter and dispersant, and grinding the mixture in a pinned-disk mill.

### 3. Dispersion Concentrate

A dispersion concentrate which is readily dispersible in water is obtained by mixing 20 parts by weight of a compound of the (I), 6 parts by weight of alkylphenol polyglycol ether (®Triton X 207), 3 parts by weight of isotridecanol polyglycol ether (8 EO) and 71 parts by weight of paraffinic mineral oil (boiling range example approx. 255 to above 277° C.), and grinding the mixture in a ball mill to a fineness of below 5 microns.

### 4. Emulsifiable Concentrate

An emulsifiable concentrate is obtained from 15 parts by weight of a compound of the formula (I), 75 parts by weight of cyclohexanone as solvent and 10 parts by weight of oxethylated nonylphenol as emulsifier.

### 5. Water-Dispersible Granules

Water-dispersible granules are obtained by mixing
75 parts by weight of a compound of the formula (I),
10 parts by weight of calcium ligninsulfonate,
5 parts by weight of sodium lauryl sulfate,
3 parts by weight of polyvinyl alcohol and
7 parts by weight of kaolin,
   grinding the mixture in a pinned-disk mill and granulating the powder in a fluidized bed by spraying on water as granulation liquid.

Water-dispersible granules are also obtained by homogenizing and precomminuting, in a colloid mill,
25 parts by weight of a compound of the formula (I),
5 parts by weight of sodium 2,2′-dinaphthylmethane-6,6′-disulfonate,
2 parts by weight of sodium oleoylmethyltauride,
1 parts by weight of polyvinyl alcohol,
17 parts by weight of calcium carbonate and
50 parts by weight of water,
   subsequently grinding the mixture in a bead mill, and atomizing and drying the resulting suspension in a spray tower by means of a single-fluid nozzle.

### C. BIOLOGICAL EXAMPLES

### 1. Pre-Emergence Herbicidal Action

Seeds of mono- and dicotyledonous weed plants are placed in sandy loam in cardboard pots and covered with soil. The compounds according to the invention, formulated as wettable powders or emulsifiable concentrates, are then applied, in the form of an aqueous suspension or emulsion, at a dosage of 320 g of active ingredient or less per hectare (converted), onto the surface of the covering earth, at an application rate of 600 to 800 l of water per ha (converted). Following treatment, the pots are placed in the greenhouse and maintained under good growth conditions for the weed plants. The visual scoring of the plant damage or emergence damage is made when the test plants have emerged, after an experimental period of 3 to 4 weeks, in comparison to untreated controls. In this experiment the compounds of Nos. 1.4, 1.19, 1.39, 2.14, 2.19, 2.39 and 3.44 for example exhibit 100% activity against Amaranthus retroflexus, Sinapis arvensis and Setaria viridis. The compounds of Nos. 1.14, 2.34 and 3.4 exhibited an activity of at least 90% against Amaranthus retroflexus and Setaria viridis.

### 2. Post-Emergence Herbicidal Action Against Weed Plants

Seeds of mono- and dicotyledonous weed plants are placed in sandy loam in cardboard pots, covered with soil and grown in the greenhouse under good growth conditions. Two to three weeks after sowing, the test plants are treated at the three-leaf stage. The compounds according to the invention, formulated as wettable powders or as emulsifiable concentrates, are sprayed at different dosages onto the surface of the green plant parts at an application rate of 600 to 800 l of water per ha (converted). After the test plants have been left to stand in the greenhouse for 3 to 4 weeks under optimal growth conditions, the activity of the compounds is scored visually. In this test, for example, the compounds of Nos. 1.39, 2.4, 3.4 and 3.39 exhibit an activity of at least 90% against Sinapis arvensis and Stellaria media at an application rate of 320 g.

### 3. Crop Plant Tolerance

In further greenhouse experiments, seeds of crop plants and of monocotyledonous and dicotyledonous weed plants are placed in sandy loam, covered with soil and placed in the greenhouse until the plants have developed two to three true leaves. Then they are treated with the compounds of the formula (I) according to the invention, and compared with those disclosed in the prior art, as described above in section 1. Four to five weeks after the application and after having been left to stand in the greenhouse, visual scoring is performed. In this test, for example, the compounds of Nos: 1.44, 2.39 and 3.44, at an application rate of 320 g, lead to no damage to maize and wheat plants.

1. A pyridinylisoxazole of the formula (I) or salt thereof 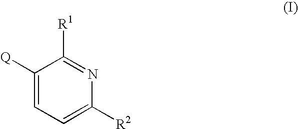
in which
Q is one of the radicals Q1, Q2 or Q3; 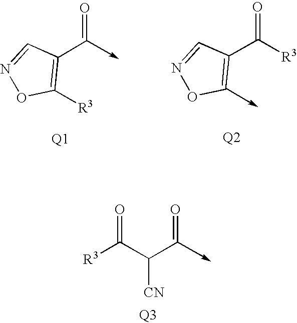
R¹ is methyl;
R² is CF₃, Cl, Br, S(O)ₙCH₃ or S(O)ₙC₂H₅;
R³ is methyl, ethyl, isopropyl, cyclopropyl or tertiary-butyl;
n is 0, 1 or 2.

2. A pyridinylisoxazole as claimed in claim 1 in which Q is Q1.

3. A pyridinylisoxazole as claimed in claim 1 in which R³ is cyclopropyl.

4. A herbicidal composition comprising a herbicidal amount of at least one compound of the formula (I) as claimed in claim 1.

5. The herbicidal composition as claimed in claim 4 as a mixture with formulating auxiliaries.

6. A method of controlling unwanted plants, which comprises applying to the plants or to the locus of unwanted plant growth an effective amount of at least one compound of the formula (I) as claimed in claim 1 or of a herbicidal composition as claimed in claim 4.

7. The use of a compound of the formula (I) according claim 1 or of a herbicidal composition as claimed in claim 4 for controlling unwanted plants.

8. The use as claimed in claim 7, wherein the compound of the formula (I) is used for controlling unwanted plants in crops of useful plants.

9. The use as claimed in claim 8, wherein the useful plants are transgenic plants.
